(19) United States
(12) Patent Application Publication
(10) Pub. No.: US 2012/0071663 A1
(43) Pub. Date: Mar. 22, 2012

(54) Preparation of N'-(4--2,5-dimethylphenyl)-N- ethyl-N-methylimidoformamide

(75) Inventors: Frank VOLZ, Koln (DE); Thomas Himmler, Odenthal (DE); Klaus Kunz, Dusseldorf (DE); Jörg Greul, Leverkusen (DE); Ulrich Heinemann, Leichlingen (DE); Thomas Seitz, Langenfeld (DE); Pierre Cristau, Lyon (FR); Oswald Ort, Leverkusen (DE)

(73) Assignee: Bayer CropScience AG, Monheim (DE)

(21) Appl. No.: 13/206,769

(22) Filed: Aug. 10, 2011

Related U.S. Application Data
(60) Provisional application No. 61/372,623, filed on Aug. 11, 2010.

(30) Foreign Application Priority Data
Aug. 10, 2010 (EP) 10172423.5

(51) Int. Cl. C07D 285/08; C07C 251/24

(52) U.S. Cl. 548/129; 564/276; 548/128

(57) ABSTRACT

The present invention relates to various processes for the preparation of N′-(4-{[3-(4-chloro-benzyl)-1,2,4-thiadiazol-5-yl]oxy}-2,5-dimethylphenyl)-N-ethyl-N-methylimidoformamide.

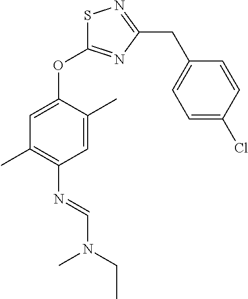

The present invention relates to different processes for the preparation of N′-(4-{[3-(4-chloro-benzyl)-1,2,4-thiadiazol-5-yl]oxy}-2,5-dimethylphenyl)-N-ethyl-N-methylimidoformamide.
WO-A-00/046 184 discloses the use of amidines as fungicides.
WO-A-03/093 224 discloses the use of arylamidine derivatives as fungicides.
WO-A-03/024 219 discloses fungicide compositions comprising at least one N2-phenylamidine derivative in combination with a further selected known active ingredient.
WO-A-04/037 239 discloses fungicidal medicaments based on N2-phenylamidine derivatives.
WO-A-07/031,513 discloses thiadiazolyl-substituted phenylamidines and their preparation and use as fungicides.
WO-A-09/156,098 discloses thiadiazolyloxyphenylamidines and their preparation and use as fungicides.

The object of the present invention is to indicate preparation routes for N′-(4-{[3-(4-chlorobenzyl)-1,2,4-thiadiazol-5-yl]oxy}-2,5-dimethylphenyl)-N-ethyl-N-methylimidoformamide.

Surprisingly, the object was achieved by a preparation process comprising at least one of the following steps (a) to (j):
(a) reaction of nitrobenzene derivatives of the formula (III) with a thiadiazolyl alcohol of the formula (II) according to the following reaction scheme:

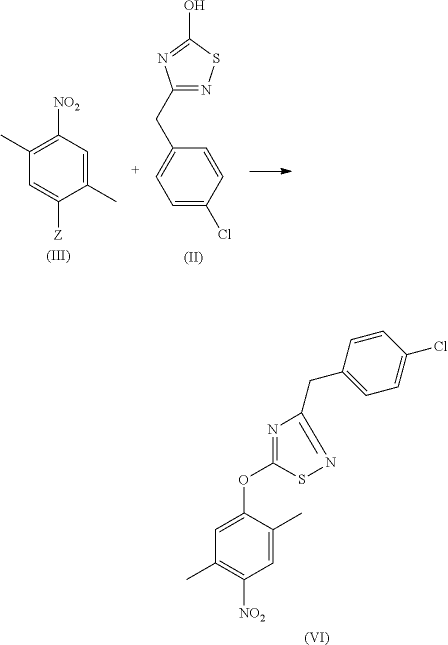

(b) reaction of nitrophenol derivatives of the formula (V) with thiadiazolyl derivatives of the formula (IV) according to the following reaction scheme:

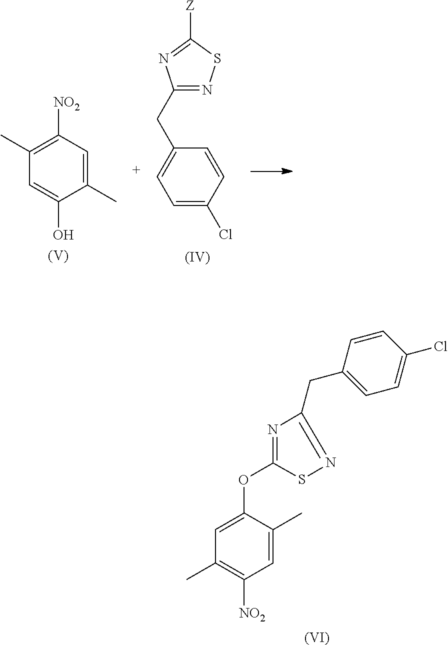

(c) reaction of anilines of the formula (VII) with a thiadiazolyl alcohol of the formula (II) according to the following reaction scheme:

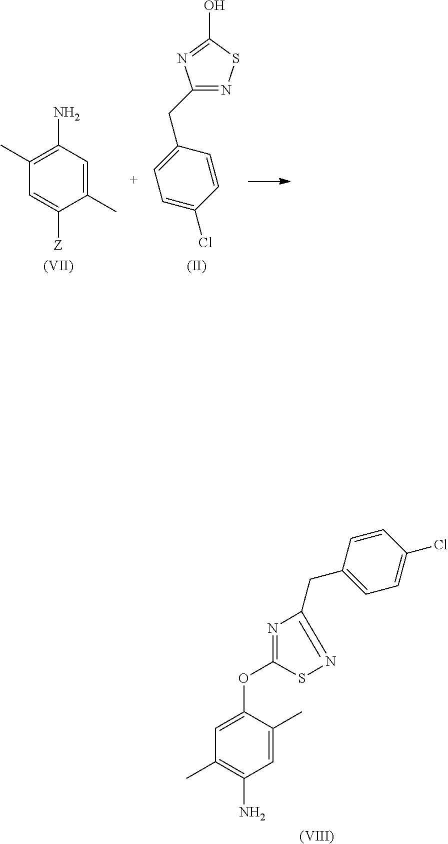

(d) reaction of aminophenols of the formula (XII) with thiadiazolyl derivatives of the formula (IV) according to the following reaction scheme:

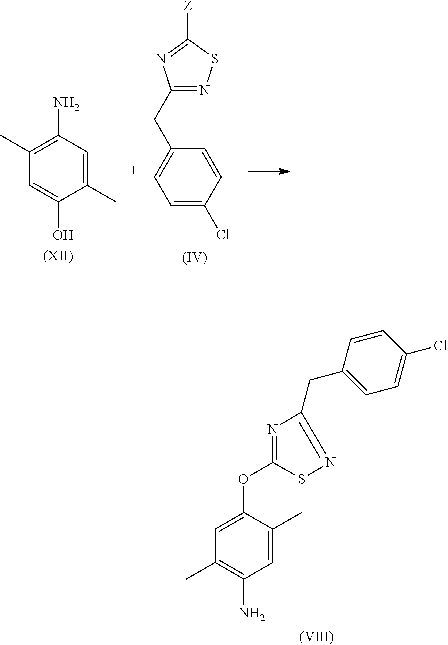

(e) reduction of the nitrophenoxy ethers of the formula (VI) to give aniline ethers of the formula (VIII) according to the following reaction scheme:

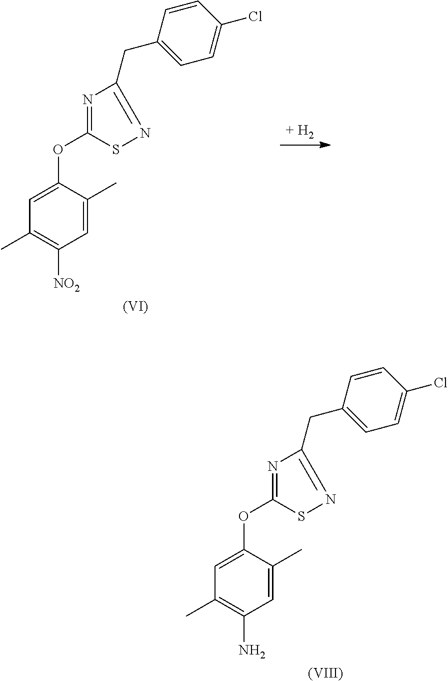

(f) reaction of the aniline ethers of the formula (VIII) with
   (i) aminoacetals of the formula (XIII) or
   (ii) amides of the formula (XIV) or
   (iii) amines of the formula (XV) in the presence of ortho esters of the formula (XVI)
   (iv) ortho esters of the formula (XVI) to give imidoformates of the formula (XVIII) and in a second step with methylethylamine (XV)
   according to the following reaction scheme:

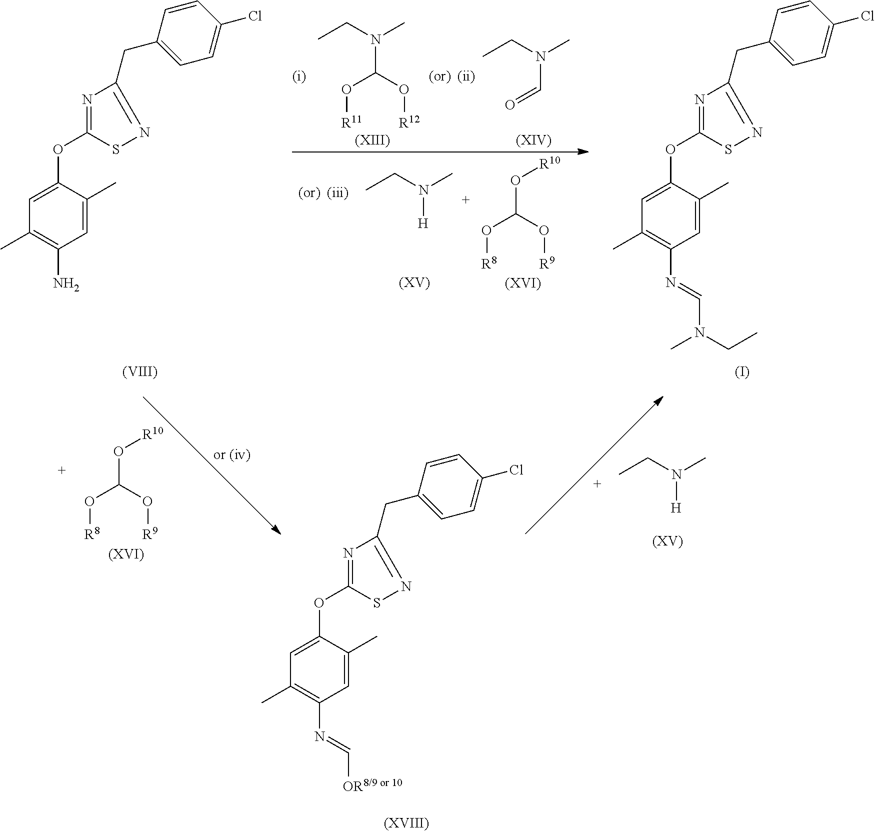

(g) reaction of the aminophenols of the formula (XII) with
   (i) aminoacetals of the formula (XIII) or
   (ii) amides of the formula (XIV) or
   (iii) amines of the formula (XV) in the presence of ortho esters of the formula (XVI)
   (iv) ortho esters of the formula (XVI) to give imidoformates of the formula (XIX) and in a second step with methylethylamine (XV)
   according to the following reaction scheme:

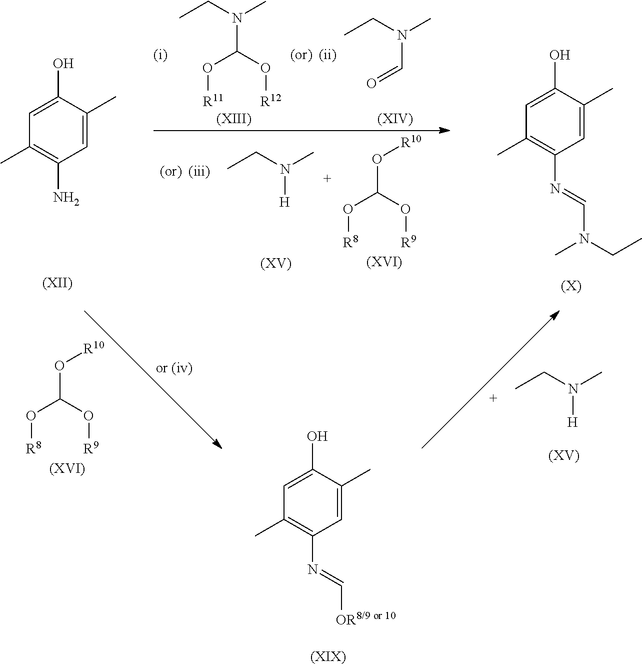

(h) reaction of the anilines of the formula (VII) with
   (i) aminoacetals of the formula (XIII) or
   (ii) amides of the formula (XIV) or
   (iii) amines of the formula (XV) in the presence of ortho esters of the formula (XVI)
   (iv) ortho esters of the formula (XVI) to give imidoformates of the formula (XX) and in a second step with methylethylamine (XV)
   according to the following reaction scheme:

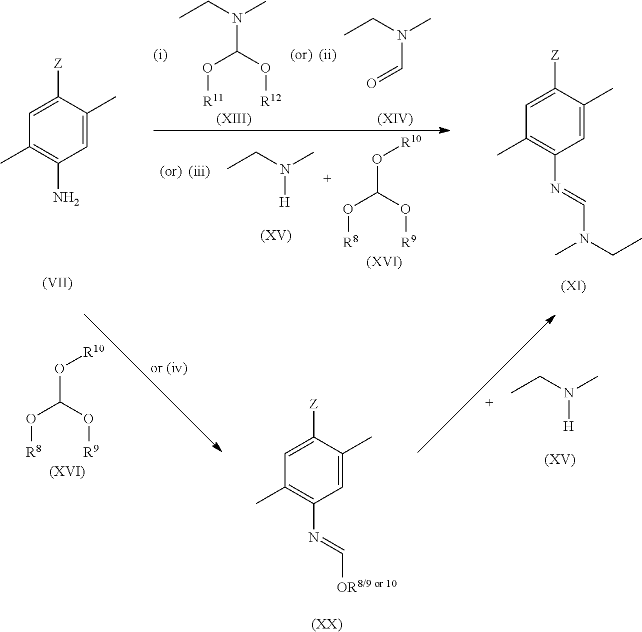

(i) reaction of amidines of the formula (XI) with a thiadiazolyl alcohol of the formula (II) according to the following reaction scheme:

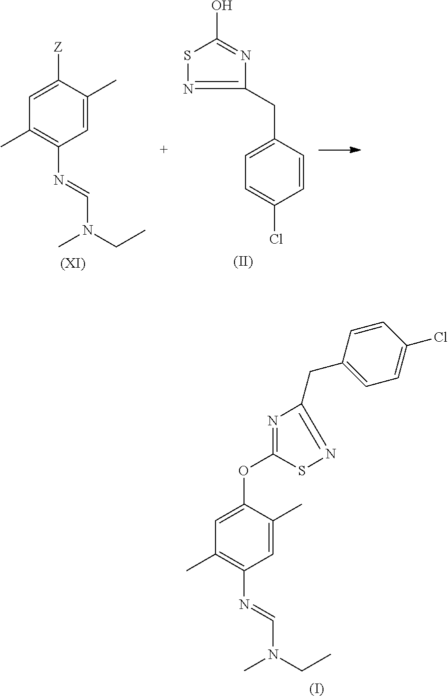

(j) reaction of amidines of the formula (X) with thiadiazolyl derivatives of the formula (IV) according to the following reaction scheme:

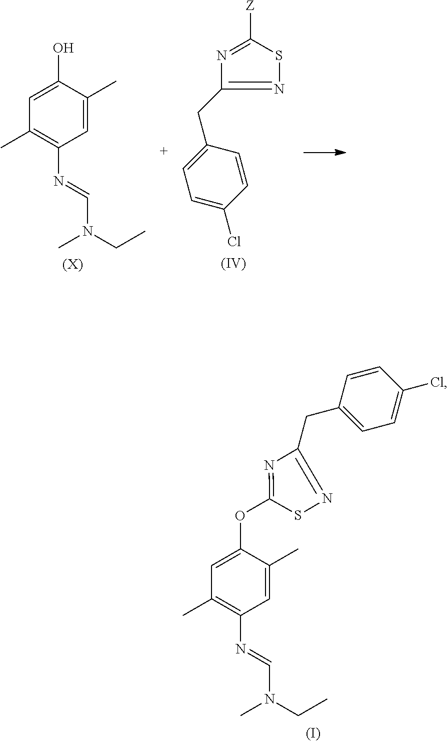

where, in the above schemes,
Z is a leaving group;
and
R⁸ to R¹⁰ independently of one another, are selected from the group consisting of hydrogen, C₁₋₁₂-alkyl, C₂₋₁₂-alkenyl, C₂₋₁₂-alkynyl, C₅₋₁₅-aryl, C₇₋₁₉-aryl-alkyl or C₇₋₁₉-alkylaryl groups and in each case R⁸ with R⁹, R⁸ with R¹⁰ or R⁹ with R¹⁰, together with the atoms to which they are bonded and optionally with further C, N, O or S atoms, can form a five-, six- or seven-membered ring;
   In a preferred embodiment of the invention, R⁸ to R¹⁰, independently of one another, are selected from the group consisting of hydrogen, C₁₋₁₂-alkyl. In a particularly preferred embodiment of the invention, R⁸ to R¹⁰ is in each case a methyl, ethyl, propyl or isopropyl group.
R¹¹ and R¹² independently of one another, are selected from the group consisting of hydrogen, C₁₋₁₂-alkyl, C₂₋₁₂-alkenyl, C₂₋₁₂-alkynyl, C₅₋₁₈-aryl or C₇₋₁₉-arylalkyl groups and, together with the atoms to which they are bonded, can form a five-, six- or seven-membered ring.

The present invention also comprises imidoformates of the formula (XVIII)

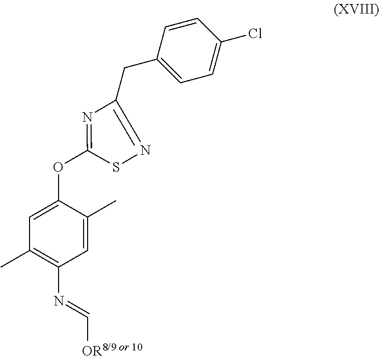

where R⁸, R⁹ and R¹⁰ have the same meaning as given above.

The present invention further comprises imidoformates of the formula (XIX)

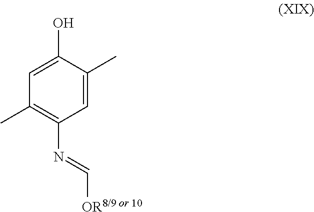

where R⁸, R⁹ and R¹⁰ have the same meaning as given above.

### GENERAL DEFINITIONS

In connection with the present invention, unless defined otherwise, alkyl groups are linear, branched or annular hydrocarbon groups which can optionally have one, two or more single or double unsaturations or one, two or more heteroatoms which are selected from O, N, P and S. Moreover, the alkyl groups according to the invention can optionally be substituted by further groups which are selected from —R′, halogen (—X), alkoxy (—OR′), thioether or mercapto (—SR′), amino (—NR′₂), silyl (—SiR′₃), carboxyl (—COOR′), cyano (—CN), acyl (—(C═O)R′) and amide groups (—CONR′₂), where R′ is hydrogen or a C₁₋₁₂-alkyl group, preferably C₂₋₄₀-alkyl group, particularly preferably C₃₋₈-alkyl group which can have one or more heteroatoms selected from N, O, P and S.

The definition of C₁-C₁₂-alkyl comprises the largest range defined herein for an alkyl group. Specifically, this definition comprises, for example, the meanings methyl, ethyl, n-propyl, isopropyl, n-butyl, isobutyl, sec-butyl and tert-butyl, n-pentyl, n-hexyl, 1,3-dimethylbutyl, 3,3-dimethylbutyl, n-heptyl, n-nonyl, n-decyl, n-undecyl, n-dodecyl.

In connection with the present invention, unless defined otherwise, alkenyl groups are linear, branched or annular hydrocarbon groups which contain at least one single unsaturation (double bond) and can optionally have one, two or more single or double unsaturations or one, two or more heteroatoms which are selected from O, N, P and S. Moreover, the alkenyl groups according to the invention can optionally be substituted by further groups which are selected from —R′, halogen (—X), alkoxy (—OR′), thioether or mercapto (—SR′), amino (—NR′₂), silyl (—SiR′₃), carboxyl (—COOR′), cyano (—CN), acyl (—(C═O)R′) and amide groups (—CONR′₂), where R′ is hydrogen or a C₁₋₁₂-alkyl group, preferably C₂₋₁₀-alkyl group, particularly preferably C₃₋₈-alkyl group which can have one or more heteroatoms selected from N, O, P and S.

The definition of C₂-C₁₂-alkenyl comprises the largest range defined herein for an alkenyl group. Specifically, this definition comprises, for example, the meanings vinyl; allyl (2-propenyl), isopropenyl (1-methylethenyl); but-1-enyl (crotyl), but-2-enyl, but-3-enyl; hex-1-enyl, hex-2-enyl, hex-3-enyl, hex-4-enyl, hex-5-enyl; hept-1-enyl, hept-2-enyl, hept-3-enyl, hept-4-enyl, hept-5-enyl, hept-6-enyl; oct-1-enyl, oct-2-enyl, oct-3-enyl, oct-4-enyl, oct-5-enyl, oct-6-enyl, oct-7-enyl; non-1-enyl, non-2-enyl, non-3-enyl, non-4-enyl, non-5-enyl, non-6-enyl, non-7-enyl, non-8-enyl; dec-1-enyl, dec-2-enyl, dec-3-enyl, dec-4-enyl, dec-5-enyl, dec-6-enyl, dec-7-enyl, dec-8-enyl, dec-9-enyl; undec-1-enyl, undec-2-enyl, undec-3-enyl, undec-4-enyl, undec-5-enyl, undec-6-enyl, undec-7-enyl, undec-8-enyl, undec-9-enyl, undec-10-enyl; dodec-1-enyl, dodec-2-enyl, dodec-3-enyl, dodec-4-enyl, dodec-5-enyl, dodec-6-enyl, dodec-7-enyl, dodec-8-enyl, dodec-9-enyl, dodec-10-enyl, dodec-11-enyl; buta-1,3-dienyl, penta-1,3-dienyl.

In connection with the present invention, unless defined otherwise, alkynyl groups are linear, branched or annular hydrocarbon groups which contain at least one double unsaturation (triple bond) and can optionally have one, two or more single or double unsaturations or one, two or more heteroatoms which are selected from O, N, P and S. Moreover, the alkynyl groups according to the invention can be optionally substituted by further groups which are selected from —R′, halogen (—X), alkoxy (—OR′), thioether or mercapto (—SR′), amino (—NR′₂), silyl (—SiR′₃), carboxyl (—COOR′), cyano (—CN), acyl (—(C═O)R′) and amide groups (—CONR′₂), where R′ is hydrogen or a linear, branched or cyclic C₁₋₁₂-alkyl group which can have one or more heteroatoms selected from N, O, P and S.

The definition of C₂-C₁₂-alkynyl comprises the largest range defined herein for an alkynyl group. Specifically, this definition comprises, for example, the meanings ethynyl (acetylenyl); prop-1-ynyl and prop-2-ynyl.

In connection with the present invention, unless defined otherwise, aryl groups are aromatic hydrocarbon groups which can have one, two or more heteroatoms which are selected from O, N, P and S and can be optionally substituted by further groups which are selected from —R′, halogen (—X), alkoxy (—OR′), thioether or mercapto (—SR′), amino (—NR′₂), silyl (—SiR′₃), carboxyl (—COOR′), cyano (—CN), acyl (—(C═O)R′) and amide groups (—CONR′₂), where R′ is hydrogen or a C₁₋₁₂-alkyl group, preferably C₂₋₁₀-alkyl group, particularly preferably C₃₋₈-alkyl group which can have one or more heteroatoms selected from N, O, P and S.

The definition of C₅₋₁₈-aryl comprises the largest range defined herein for an aryl group having 5 to 18 backbone atoms, where the carbon atoms may be exchanged for heteroatoms. Specifically, this definition comprises, for example, the meanings cyclopentadienyl, phenyl, cycloheptatrienyl, cyclooctatetraenyl, naphthyl and anthracenyl; 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl; 1-pyrrolyl, 1-pyrazolyl, 1,2,4-triazol-1-yl, 1-imidazolyl, 1,2,3-triazol-1-yl, 1,3,4-triazol-1-yl; 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

In connection with the present invention, unless defined otherwise, arylalkyl groups (aralkyl groups) are alkyl groups which are substituted by aryl groups and which can have a C₁₋₈-alkylene chain and, in the aryl backbone or the alkylene chain, may be substituted by one or more heteroatoms which are selected from O, N, P and S and optionally by further groups which are selected from —R′, halogen (—X), alkoxy (—OR′), thioether or mercapto (—SR′), amino (—NR′₂), silyl (—SiR′₃), carboxyl (—COOR′), cyano (—CN), acyl (—(C═O)R′) and amide groups (—CONR′₂), where R′ is hydrogen or a C₁₋₁₂-alkyl group, preferably C₂₋₁₀-alkyl group, particularly preferably C₃₋₈-alkyl group which can have one or more heteroatoms selected from N, O, P and S.

The definition of C₇₋₁₉-aralkyl group comprises the largest range defined herein for an arylalkyl group having in total 7 to 19 carbon atoms in the backbone and alkylene chain. Specifically, this definition comprises, for example, the meanings benzyl and phenylethyl.

In connection with the present invention, unless defined otherwise, alkylaryl groups (alkaryl groups) are aryl groups which are substituted by alkyl groups and which can have a C₁₋₈-alkyl chain and, in the aryl backbone or the alkyl chain, may be substituted by one or more heteroatoms which are selected from O, N, P and S and optionally by further groups which are selected from —R′, halogen (—X), alkoxy (—OR′), thioether or mercapto (—SR′), amino (—NR′₂), silyl (—SiR′₃), carboxyl (—COOR′), cyano (—CN), acyl (—(C═O)R′) and amide groups (—CONR′₂), where R′ is hydrogen or a C₁₋₁₂-alkyl group, preferably C₂₋₁₀-alkyl group, is particularly preferably C₃₋₈-alkyl group which can have one or more heteroatoms selected from N, O, P and S.

The definition of C₇₋₁₉-alkylaryl group comprises the largest range defined herein for an alkylaryl group having in total 7 to 19 carbon atoms in the backbone and alkyl chain. Specifically, this definition comprises for example the meanings tolyl-, 2,3-, 2,4-, 2,5-, 2,6-, 3,4- or 3,5-dimethyl-phenyl.

Moreover, the alkyl, alkenyl, alkynyl, aryl, alkaryl and aralkyl groups can have one or more heteroatoms which—unless defined otherwise—are selected from N, O, P and S. The heteroatoms here replace the numbered carbon atoms.

### Preparation of N′-(4-{[3-(4-chlorobenzyl)-1,2,4-thiadiazol-5-yl]oxy}-2,5-dimethylphenyl)-N-ethyl-N-methylimidoformamide

N′-(4-{[3-(4-Chlorobenzyl)-1,2,4-thiadiazol-5-yl]oxy}-2,5-dimethylphenyl)-N-ethyl-N-methyl-imidoformamide can be obtained by the process shown in the following schemes (Ia) and (Ib):

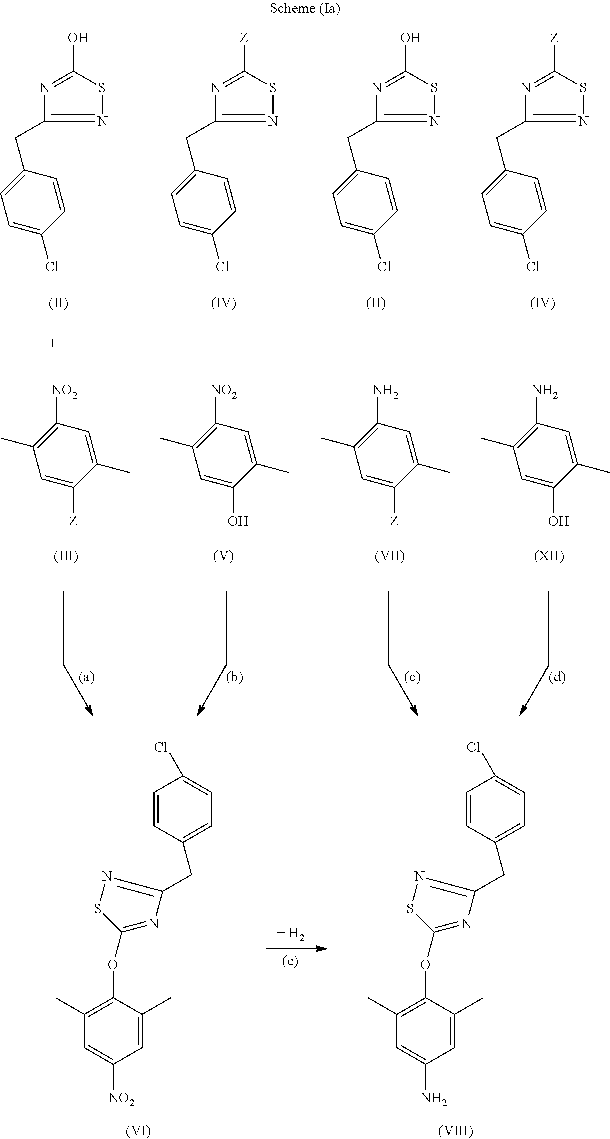

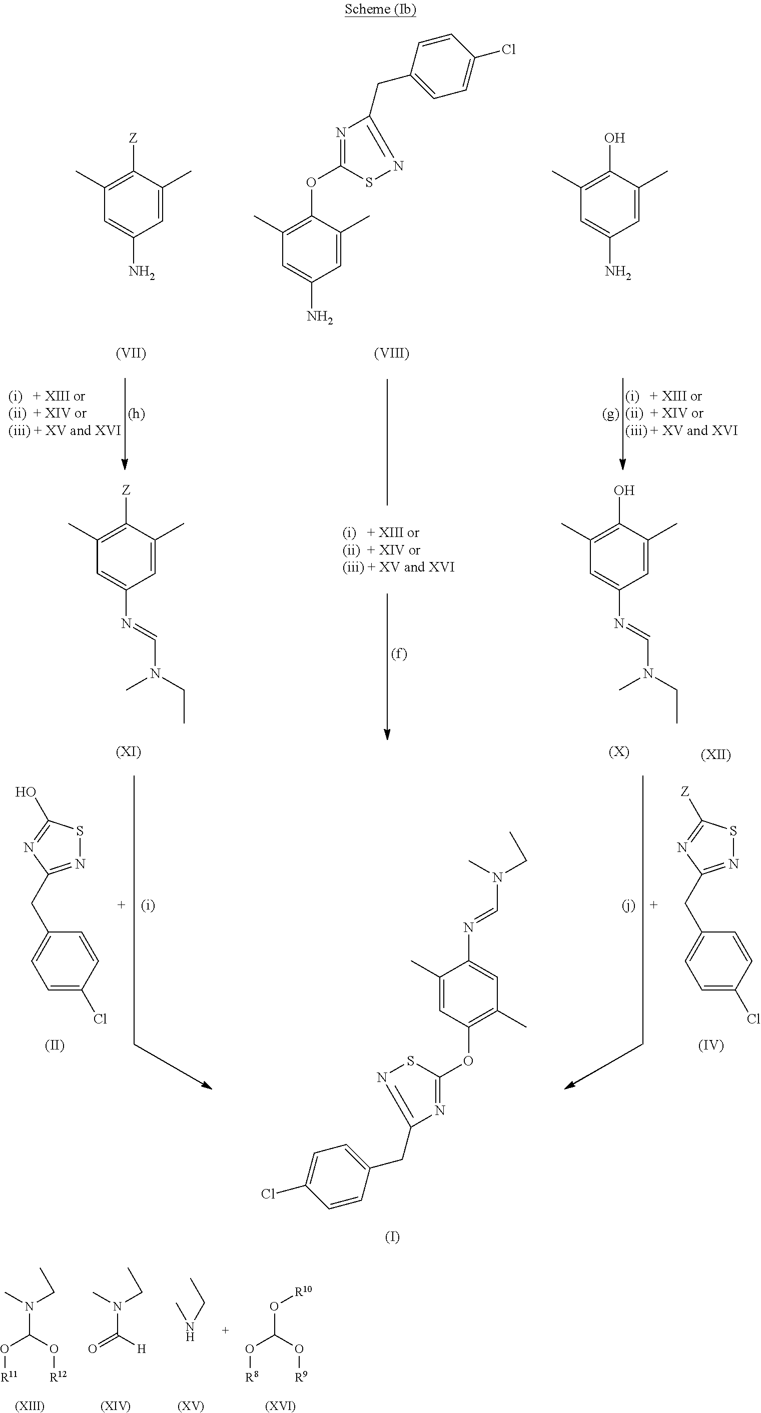

### Step (a)

In one embodiment according to the invention, nitrobenzene derivatives of the formula (III) are reacted with a thiadiazolyl alcohol of the formula (II) according to the following reaction scheme to give a nitrophenyl ether of the formula (VI):

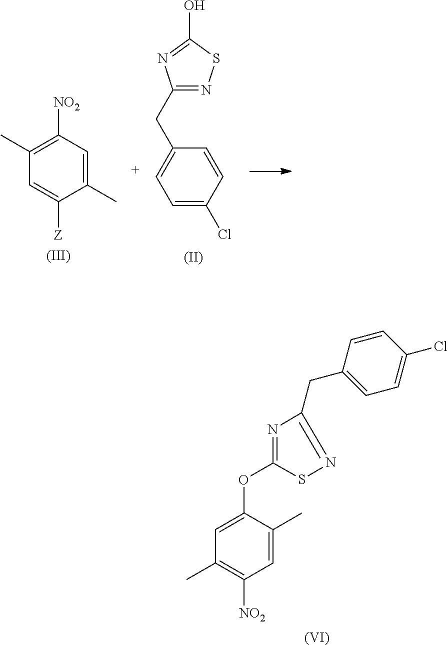

Suitable leaving groups Z are all substituents which have an adequate nucleofugicity under the prevailing reaction conditions. For example, mention may be made of halogens, triflate, mesylate, tosylate or SO₂Me as suitable leaving groups.

The nitrobenzene derivatives of the formula (III) are available as per Journal of the Chemical Society 1926, 2036.

The reaction preferably takes place in the presence of a base.

Suitable bases are organic and inorganic bases which are usually used in such reactions. Preference is given to using bases which are selected, for example, from the group consisting of hydrides, hydroxides, amides, alcoholates, acetates, fluorides, phosphates, carbonates and hydrogen-carbonates of alkali metals or alkaline earth metals. Particular preference here is given to sodium amide, sodium hydride, lithium diisopropylamide, sodium methanolate, potassium tert-butanolate, sodium hydroxide, potassium hydroxide, sodium acetate, sodium phosphate, potassium phosphate, potassium fluoride, caesium fluoride, sodium carbonate, potassium carbonate, potassium hydrogen-carbonate, sodium hydrogencarbonate and caesium carbonate. Moreover, it is also possible to use tertiary amines, such as e.g. trimethylamine, triethylamine, tributylamine, N,N-dimethylaniline, N,N-dimethylbenzylamine, N-methylpiperidine, N,N-dimethylaminopyridine, diazabicyclooctane (DABCO), diazabicyclononene (DBN and diazabicycloundecene (DBU) and also pyridine, 2-, 3- or 4-picoline, 2,3-, 2,4-, 2,5-, 2,6-, 3,4- and 3,5-lutidine and 5-ethyl-2-methylpyridine.

If necessary, a catalyst, which is selected from the group consisting of palladium, copper and salts or complexes thereof, can be used.

The reaction of the nitrobenzene derivative with the phenol can take place without dilution or in a solvent; preferably, the reaction is carried out in a solvent which is selected from customary solvents that are inert under the prevailing reaction conditions.

Preference is given to aliphatic, alicyclic or aromatic hydrocarbons, such as, for example, petroleum ether, hexane, heptane, cyclohexane, methylcyclohexane, benzene, toluene, xylene or decalin; halogenated hydrocarbons, such as e.g. chlorobenzene, dichlorobenzene, dichloromethane, chloroform, tetrachloromethane, dichloroethane or trichloroethane; ethers, such as, for example, diethyl ether, diisopropyl ether, methyl tert-butyl ether (MTBE), methyl tert-amyl ether, dioxane, tetrahydrofuran, 1,2-dimethoxyethane, 1,2-diethoxyethane or anisole; nitriles, such as, for example, acetonitrile, propionitrile, n- or isobutyronitrile or benzonitrile; amides, such as, for example, N,N-dimethylformamide (DMF), N,N-dimethylacetamide, N-methylformanilide, N-methyl-pyrrolidone (NMP) or hexamethylphosphoramide; or mixtures of these with water, and also pure water.

The reaction can be carried out in vacuo, at atmospheric pressure or under superatmospheric pressure and at temperatures of from −20 to 200° C., the reaction preferably takes place at atmospheric pressure and at temperatures of from 50 to 150° C.

### Step (b)

In an alternative embodiment according to the invention, the nitrophenol derivative of the formula (V) is reacted with thiadiazolyl derivatives of the formula (IV) according to the following reaction scheme to give a nitrophenyl ether of the formula (VI):

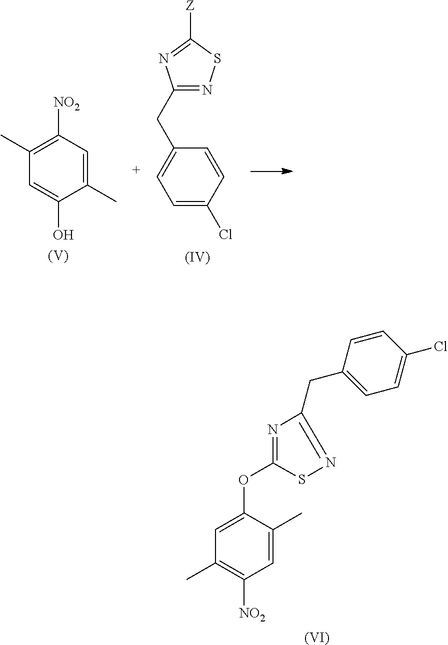

The nitrophenol derivative of the formula (V) is obtainable as per Journal of the Chemical Society 1926, 2036.

As regards reaction conditions, solvents, catalysts and suitable leaving groups, reference may be made to step (a).

### Step (c)

In a further alternative embodiment according to the invention, anilines of the formula (VII) are reacted with a thiadiazolyl alcohol of the formula (II) according to the following reaction scheme to give the aminophenyl ether of the formula (VIII):

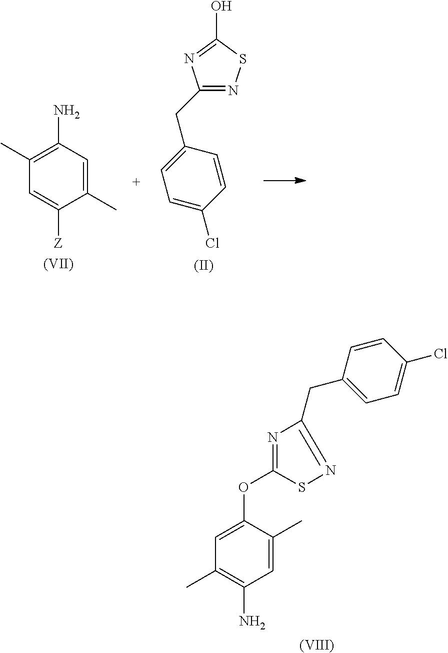

With regard to reaction conditions, solvents, catalysts and suitable leaving groups, reference may be made to step (a).

### Step (d)

In a further alternative embodiment according to the invention, the aminophenol of the formula (XII) is reacted with thiadiazolyl derivatives of the formula (IV) according to the following reaction scheme to give the aminophenyl ether of the formula (VIII):

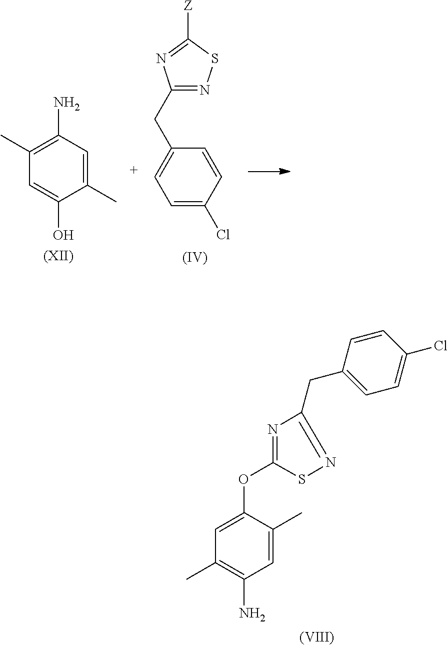

With regard to reaction conditions, solvents, catalysts and suitable leaving groups, reference may be made to steps (a) and (c).

### Step (e)

The nitrophenyl ether of the formula (VI) obtained in steps (a) and (b) can be reduced according to the following reaction scheme to give the aniline ether of the formula (VIII):

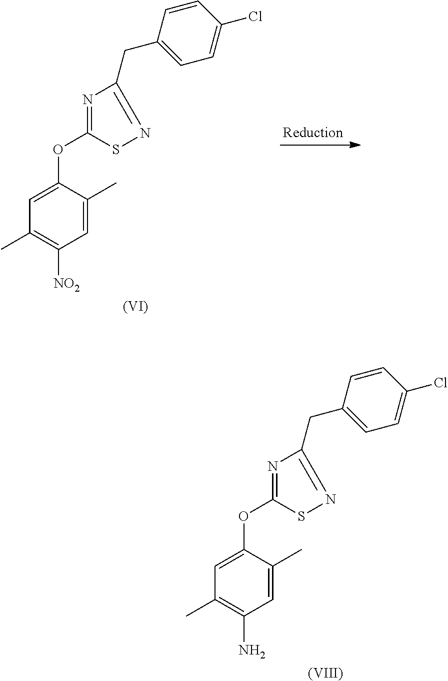

The reduction according to step (e) can take place by any of the methods described in the prior art for reducing nitro groups.

Preferably, the reduction takes place with tin chloride in concentrated hydrochloric acid, as described in WO-A-0 046 184. Alternatively, the reduction can, however, also take place with hydrogen gas, optionally in the presence of suitable hydrogenation catalysts, such as e.g. Raney nickel or Pd/C. The reaction conditions have already been described in the prior art and are familiar to the person skilled in the art.

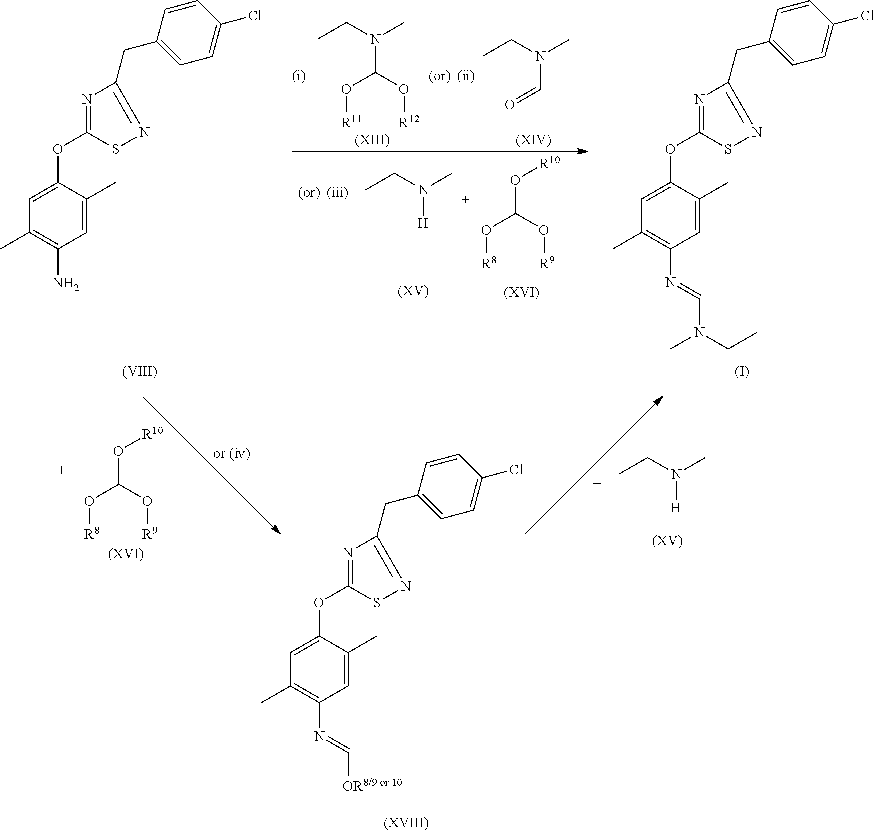

The individual alternative embodiments (i) to (iii) of the process according to the invention will be explained below in brief:
(i) according to one embodiment of the invention, which is shown in scheme (Ib) as step (i), the aniline ethers of the formula (VIII) are reacted with aminoacetals of the formula (XIII), in which R¹¹ and R¹² are selected from C₁₋₈-alkyl groups, preferably from C₂₋₆-alkyl groups, particularly preferably from C₃₋₅-alkyl groups and, together with the O atoms to which they are bonded, can form a five- or six-membered ring, to give the compound of the formula (I) according to the invention.

The aminoacetals of the formula (XIII) are obtainable from the formamides described in JACS, 65, 1566 (1943) by reaction with alkylating reagents, such as e.g. dimethyl sulphate.

The reaction according to step (i) preferably takes place in the presence of an acid.

Suitable acids are selected, for example, from the group consisting of organic and inorganic acids, with p-toluenesulphonic acid, methanesulphonic acid, hydrochloric acid (gaseous, aqueous or in organic solution) or sulphuric acid being preferred.
(ii) In an alternative embodiment according to the invention, which is shown in scheme (Ib) as step (ii), the aniline ether of the formula (VIII) is reacted with the amide of the formula (XIV) to give the compound of the formula (I) according to the invention.
   The reaction according to step (ii) optionally takes place in the presence of a halogenating agent. Suitable halogenating agents are selected, for example, from the group consisting of PCl₅, PCl₃, POCl₃ or SOCl₂.
   Moreover, the reaction can alternatively take place in the presence of a condensing agent.
   Suitable condensing agents are those which are usually used for the linking of amide bonds; by way of example, mention may be made of acid halide formers such as e.g. phosgene, phosphorus tribromide, phosphorus trichloride, phosphorus pentachloride, phosphorus trichloride oxide or thionyl chloride; anhydride formers such as e.g. chloroformates such as e.g. methyl chloroformate, isopropyl chloroformate, isobutyl chloroformate, methanesulphonyl chloride; carbodiimides such as e.g. N,N′-dicyclohexyl-carbodiimide (DCC) or other customary condensing agents such as e.g. phosphorus pentoxide, polyphosphoric acid, N,N′-carbodiimidazole, 2-ethoxy-N-ethoxycarbonyl-1,2-dihydroquinoline (EEDQ), triphenylphosphine/tetrachloromethane or bromotri-pyrrolidinophosphonium hexafluorophosphate.
   The reaction according to step (ii) preferably takes place in a solvent which is selected from the customary solvents that are inert under the prevailing reaction conditions. Preference is given to using aliphatic, alicyclic or aromatic hydrocarbons, such as, for example, petroleum ether, hexane, heptane, cyclohexane, methylcyclohexane, benzene, toluene, xylene or decalin; halogenated hydrocarbons, such as e.g. chlorobenzene, dichlorobenzene, dichloromethane, chloroform, tetrachloromethane, dichloroethane or trichloroethane; ethers, such as, for example, diethyl ether, diisopropyl ether, methyl tert-butyl ether (MTBE), methyl tert-amyl ether, dioxane, tetrahydrofuran, 1,2-dimethoxy-ethane, 1,2-diethoxyethane or anisole; nitriles, such as, for example, acetonitrile, propionitrile, n- or isobutyronitrile or benzonitrile; amides, such as, for example, N,N-dimethylformamide (DMF), N,N-dimethylacetamide, N-methylformanilide, N-methylpyrrolidone (NMP) or hexamethylphosphoramide; esters, such as, for example, methyl or ethyl acetate; sulphoxides, such as, for example, dimethyl sulphoxide (DMSO); sulphones, such as, for example, sulpholane; alcohols, such as, for example, methanol, ethanol, n- or isopropanol, n-, iso-, sec- or tert-butanol, ethanediol, propane-1,2-diol, ethoxyethanol, methoxyethanol, diethylene glycol monomethyl ether, diethylene glycol monoethyl ether or mixtures of these.
(iii) According to a further alternative embodiment according to the invention, which is shown in scheme (Ib) as step (iii), the aniline ether of the formula (VIII) is reacted with an amine of the formula (XV) in the presence of orthoesters of the formula (XVI), in which R⁸ to R¹⁰, independently of one another, are selected from C₁₋₈-alkyl groups, particularly preferably from C₁₋₄-alkyl groups and, together with the O atoms to which they are bonded, can form a five- or six-membered ring, to give the compound of the formula (I) according to the invention.
   The reaction according to step (iii) preferably takes place in a solvent which is selected from the customary solvents that are inert under the prevailing reaction conditions. Preference is given to using aliphatic, alicyclic or aromatic hydrocarbons, such as, for example, petroleum ether, hexane, heptane, cyclohexane, methylcyclohexane, benzene, toluene, xylene or decalin; halogenated hydrocarbons, such as e.g. chlorobenzene, dichlorobenzene, dichloromethane, chloroform, tetrachloromethane, dichloroethane or trichloroethane; ethers, such as, for example, diethyl ether, diisopropyl ether, methyl tert-butyl ether (MTBE), methyl tert-amyl ether, dioxane, tetrahydrofuran, 1,2-dimethoxyethane, 1,2-diethoxyethane or anisole; nitriles, such as, for example, acetonitrile, propionitrile, n- or isobutyronitrile or benzonitrile; amides, such as, for example, N,N-dimethylformamide (DMF), N,N-dimethylacetamide, N-methylformanilide, N-methylpyrrolidone (NMP) or hexamethylphosphoramide; esters, such as, for example, methyl or ethyl acetate; sulphoxides, such as, for example, dimethyl sulphoxide (DMSO); sulphones, such as, for example, sulpholane; alcohols, such as, for example, methanol, ethanol, n- or isopropanol, n-, iso-, sec- or tert-butanol, ethanediol, propane-1,2-diol, ethoxyethanol, methoxyethanol, diethylene glycol monomethyl ether, diethylene glycol monoethyl ether; or mixtures of these with water, and also pure water.
(iii) Alternatively to the process (iii), an imidoformate of the formula (XVIII) can be prepared by adding an orthoester of the formula (XVI) to an aniline ether of the formula (VIII). The imidoformate of the formula (XVIII) is then reacted in a second step with an amine of the formula (XV) to give the compound of the formula (I) according to the invention.

### Step (g)

In an alternative embodiment according to the invention, the aminophenol of the formula (XII) can already be reacted
(i) with aminoacetals of the formula (XIII) or
(ii) with the amide of the formula (XIV) or
(iii) with the amine of the formula (XV) in the presence of orthoesters of the formula (XVI)
(iv) orthoesters of the formula (XVI) to give imidoformates of the formula (XVIV) and in a second step with methylethylamine (XV)
   according to the following reaction scheme to give the amidine of the formula (X):

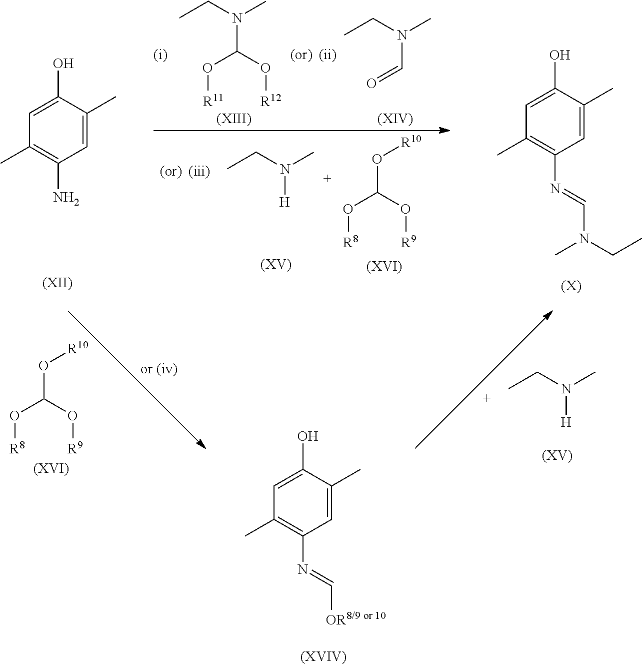

As regards the reaction conditions, solvents and catalysts, reference may be made to step (f).

The further reaction of the amidine of the formula (X) to the target molecule of the formula (I) according to the invention can take place for example as described in step (j).

### Step (h)

In an alternative embodiment according to the invention, the aminophenyl derivatives of the formula (VII) can be reacted
(i) with aminoacetals of the formula (XIII) or
(ii) with the amide of the formula (XIV) or
(iii) with the amine of the formula (XV) in the presence of orthoesters of the formula (XVI)
(iv) orthoesters of the formula (XVI) to give imidoformates of the formula (XX) and in a second step with methylethylamine (XV)
   according to the following reaction scheme to give amidines of the formula (XI):

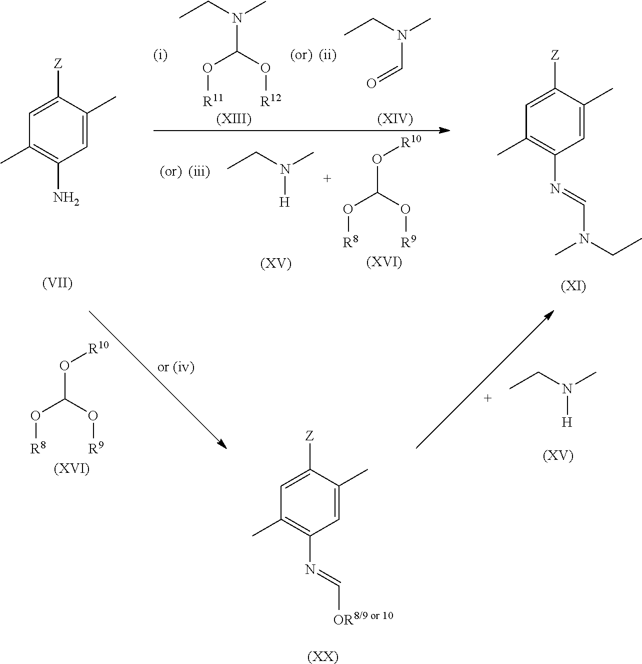

As regards the reaction conditions, solvents and catalysts, reference may be made to step (f).

The further reaction of the amidines of the formula (XI) to give the target molecule of the formula (I) according to the invention can take place, for example, as described in step (i).

### Step (i)

According to a further embodiment according to the invention, the amidines of the formula (XI) obtainable from step (h) can be reacted with the thiadiazolyl alcohol of the formula (II) to give the target molecule of the formula (I) according to the invention as per the following reaction scheme:

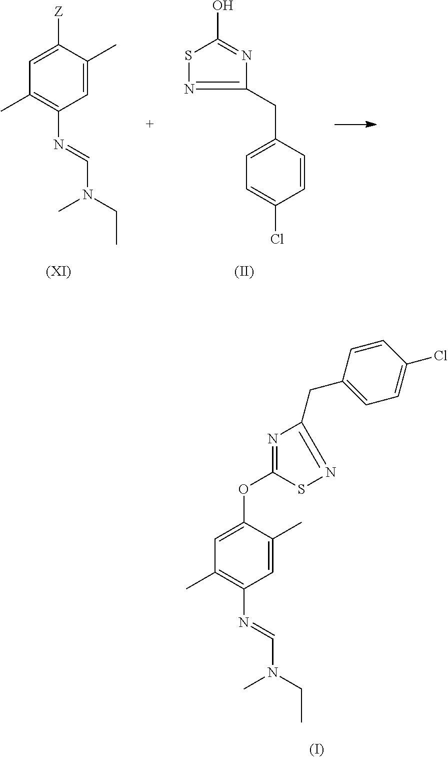

As regards the reaction conditions, solvents and catalysts, reference may be made to step (f).

### Step (j)

According to a further embodiment according to the invention, the amidine of the formula (X) obtainable from step (g) can be reacted with thiadiazolyl derivatives of the formula (IV) to give the target molecule of the formula (I) according to the invention as per the following reaction scheme:

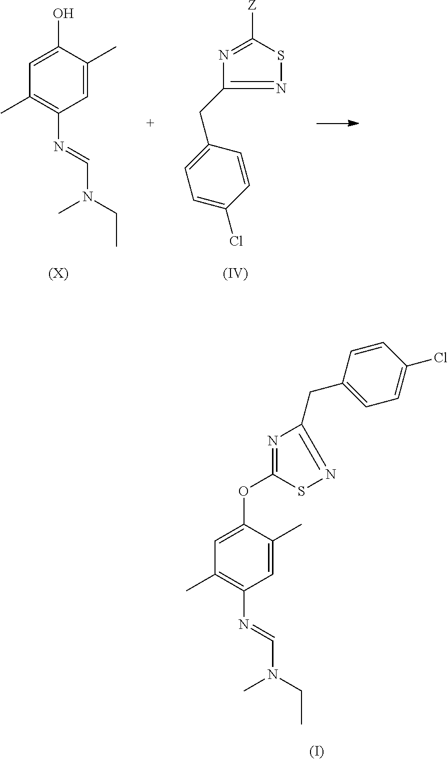

As regards the reaction conditions, solvents and catalysts, reference may be made to step (f) and to Tables I and II.

In connection with the processes according to the invention for the preparation of the amidine of the formula (I), the following combinations of reaction steps are to be considered advantageous: steps (a), (e) and (f); steps (b), (e) and (f); steps (c) and (f); steps (d) and (f); steps (h) and (i) and/or steps (g) and (j).

The preparation of the thiadiazolyloxyphenylamidine according to the invention optionally takes place without interim isolation of the intermediates.

The final purification of the thiadiazolyloxyphenylamidine can optionally take place by customary purification methods. Preferably, the purification takes place by crystallization.

The thiadiazolyl derivatives of the formula (IVa) used in steps (b), (d) and (j) of the process described above in which Z is a chlorine atom can be obtained, for example, as per the process described in the following scheme or those in DE-A-960281 or in Chemische Berichte, 90, 182-7; 1957:

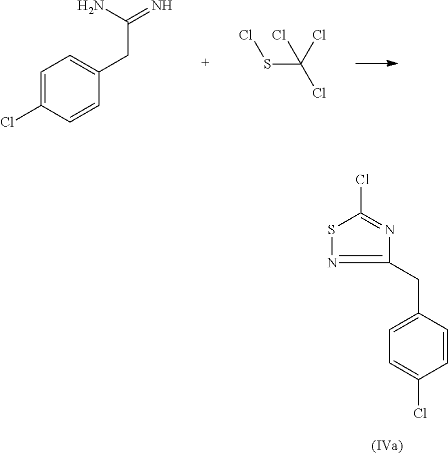

The chlorides of the formula (IVa) can be converted to the alcohols of the formula (II) by acidic hydrolysis.

The thiadiazolyl derivatives of the formula (IVb) used in steps (b), (d) and (j) of the process described above, in which Z is a tosyl group, can be obtained, for example, as per the process described in the following scheme:

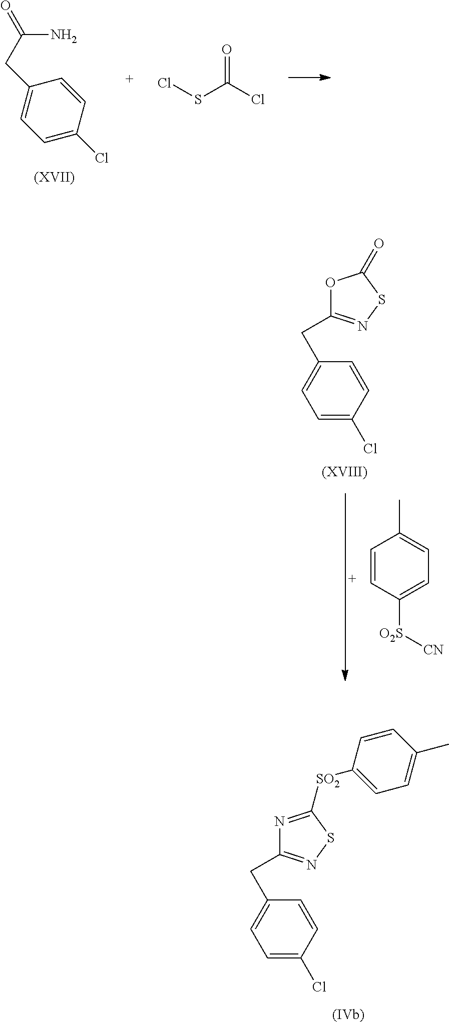

The carboxamides of the general formula (XVII) that are used can be prepared, for example, in accordance with the procedure in Houben-Weyl VIII, p. 655 ff.

### SYNTHESIS EXAMPLES

### Example of Process d)

Under an argon protective-gas atmosphere 2.74 g [20 mmol] of 4-amino-2,5-dimethylphenol and 1.2 g [30 mmol] of NaOH (in the form of so-called micropills) are introduced into 15 ml of degassed and anhydrous DMAC. The mixture is stirred for 30 minutes at 40° C., then cooled to room temperature and then, with cooling at a maximum of 20° C., a solution of 4.9 g [10 mmol] of 5-chloro-3-(4-chlorobenzyl)-1,2,4-thiadiazole in 5 ml of degassed DMAC is added dropwise. After 2 hours at room temperature, the reaction mixture is filtered over some kieselguhr and then the filtrate is concentrated on a rotary evaporator at a maximum bath temperature of 50° C. in vacuo. The oil obtained in this way is stirred with ca. 50 ml of water. Extraction is carried out with in total about 100 ml of methylene chloride, and the combined organic phases are washed with water and dried over sodium sulphate. 6.9 g of brown oil result, which, according to HPLC analysis, consists to 84% of 4-{[3-(4-chlorobenzyl)-1,2,4-thiadiazol-5-yl]oxy}-2,5-dimethylaniline (yield=83.8% of theory).

LC/MS: m/e=346 (MH⁺).

### Example of Process f)

2.38 g [20 mmol] of thionyl chloride are added dropwise to a solution of 1.045 g [12 mmol] of N-ethyl-N-methylformamide in 10 ml of methylene chloride. The mixture is heated at reflux for 2 hours and cooled to 5-10° C. and then a solution of 2.42 g [7 mmol] of 4-{[3-(4-chlorobenzyl)-1,2,4-thiadiazol-5-yl]oxy}-2,5-dimethylaniline is added dropwise. After 2 hours at 5-10° C., the mixture is allowed to reach room temperature and is then stirred for a further 2 hours at room temperature. The reaction mixture is then stirred with 30 ml of semiconcentrated hydrochloric acid. The organic phase is separated off, washed with 30 ml of water and concentrated on a rotary evaporator. This gives 3.9 g of a beige solid which, according to HPLC, consists to 90% of N′-(4-{[3-(4-chlorobenzyl)-1,2,4-thiadiazol-5-yl]oxy}-2,5-dimethylphenyl)-N-ethyl-N-methylimidoformamide hydrochloride.

### Example of Process g)

At a maximum of 60° C., 9.2 g [60 mmol] of POCl₃ are added dropwise to a solution of 6.86 g [50 mmol] of 4-amino-2,5-dimethylphenol and 5.23 g [60 mmol] of N-ethyl-N-methylformamide in 20 ml of acetonitrile. After 1 hour at 60° C., the mixture is cooled to room temperature, concentrated to give a thick slurry, which is stirred with ca. 20 ml of cold isopropanol; the solid is filtered off with suction, washed with 10 ml of cold isopropanol and dried. This gives 10.4 g of a pale beige solid which, according to GC/MS (sil.) and HPLC, consists to 99.5% of N-ethyl-N′-(4-hydroxy-2,5-dimethylphenyl)-N-methylimidoformamide hydrochloride (yield=85% of theory).

GC/MS (sil.): m/e=278 (M⁺, sil., 100%), 263 (M⁺, sil—Me, 70%), 205 (M⁺ sil—73, 20%).

### Example of Process f)

0.5 g of the ion exchange resin Amberlite IR 120 (dried prior to use by heating in vacuo) is added to a solution of 15.45 g [44.7 mmol] of 4-{[3-(4-chlorobenzyl)-1,2,4-thiadiazol-5-yl]oxy}-2,5-di-methylaniline in 100 ml of trimethyl orthoformate, and the mixture is then heated for 1 hour at about 100° C. so that the methanol which is formed can be distilled off continuously via a distillation bridge. The mixture is then filtered and the filtrate is concentrated on a rotary evaporator. This gives 19.35 g of a thick oil which, according to GC/MS, comprises to 85.8% methyl (4-{[3-(4-chlorobenzyl)-1,2,4-thiadiazol-5-yl]oxy}-2,5-dimethylphenyl)imidoformate (yield=96% of theory).

GC/MS: m/e=387 (M⁺, ³⁵Cl, 100%).

¹H-NMR (400 MHz, d6-DMSO): 7.93 (s, 1H), 7.37 (d, 2H), 7.32 (d, 2H), 7.24 (s, 1H), 6.89 (s, 1H), 4.10 (s, 1H), 3.29 (s, 3H), 2.15 (s, 3H), 2.11 (s, 3H).

### Example of Process f)

A solution of 1.84 g [5 mmol] of methyl (4-{[3-(4-chlorobenzyl)-1,2,4-thiadiazol-5-yl]oxy}-2,5-di-methylphenyl)imidoformate and 0.323 g [5.75 mmol] of ethylmethylamine in 10 ml of anhydrous methanol is left to stand for 16 hours at room temperature. The reaction mixture is then stirred into 40 ml of cold water. Extraction is carried out several times with methylene chloride, and the combined organic phases are washed with water, dried over sodium sulphate and concentrated by evaporation. This results in 2.35 g of residue which, according to HPLC, consists to 80% of N′-(4-{[3-(4-chlorobenzyl)-1,2,4-thiadiazol-5-yl]oxy}-2,5-dimethylphenyl)-N-ethyl-N-methyl-imidoformamide (yield=90% of theory).

LC/MS: m/e=415 (MH⁺).

GC/MS: m/e=414 (M⁺, ³⁵Cl, 60%), 399 (M-Me, ³⁵Cl, 100%).

### Example of Process f)

Two drops of sulphuric acid are added to a solution of 20 g [57 mmol] of 4-{[3-(4-chlorobenzyl)-1,2,4-thiadiazol-5-yl]oxy}-2,5-dimethylaniline, 8.4 g [57 mmol] of triethyl orthoformate in 100 ml of butyronitrile, and the resulting reaction mixture is stirred for 2 hours at 115° C. After cooling to 40° C., 3.8 g [62 mmol] of ethylmethylamine are added and the mixture is stirred overnight at 40° C. The entire reaction mixture is concentrated on a rotary evaporator, giving a highly viscous oil which, according to HPLC, consists to 52% of N′-(4-{[3-(4-chlorobenzyl)-1,2,4-thiadiazol-5-yl]oxy}-2,5-dimethylphenyl)-N-ethyl-N-methyl-imidoformamide (yield=47% of theory).

### Example of Process g)

2.74 g [20 mmol] of 4-amino-2,5-dimethylphenol are introduced into 20 ml of trimethyl orthoformate; 228 mg of para-toluenesulphonic acid hydrate are added and the mixture is heated at reflux for 2 hours. The reaction mixture is then concentrated by evaporation under reduced pressure, giving 4.9 g of a brown solid which, according to GC/MS analysis, consists to 61.3% of methyl (4-hydroxy-2,5-dimethylphenyl)imidoformate (83.8% of theory).

GC/MS: m/e=179 (M⁺, 80%), 148 (M⁺-OMe, 100%).

1. Process for the preparation of N′-(4-{[3-(4-chlorobenzyl)-1,2,4-thiadiazol-5-yl]oxy}-2,5-dimethylphenyl)-N-ethyl-N-methylimidoformamide comprising at least one of the following steps (a) to (j):
(a) reaction of nitrobenzene derivatives of the formula (III) with a thiadiazolyl alcohol of the formula (II) according to the following reaction scheme: 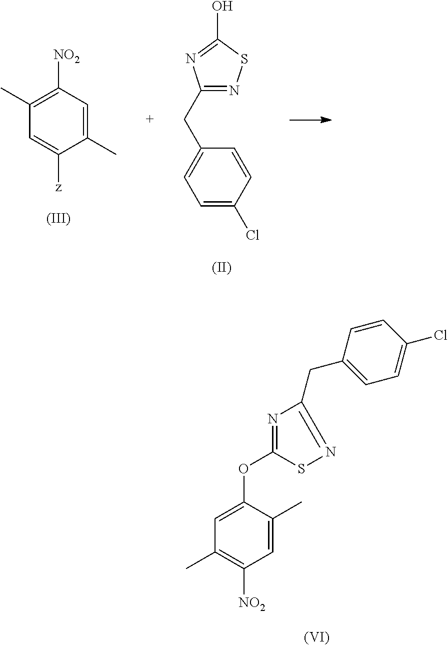
(b) reaction of the nitrophenol derivative of the formula (V) with thiadiazolyl derivatives of the formula (IV) according to the following reaction scheme: 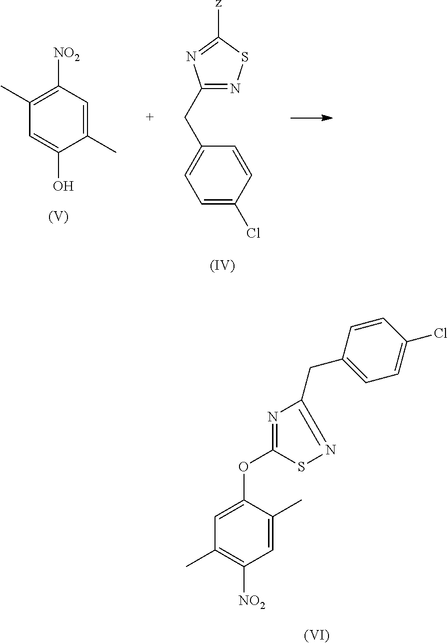
(c) reaction of anilines of the formula (VII) with a thiadiazolyl alcohol of the formula (II) according to the following reaction scheme: 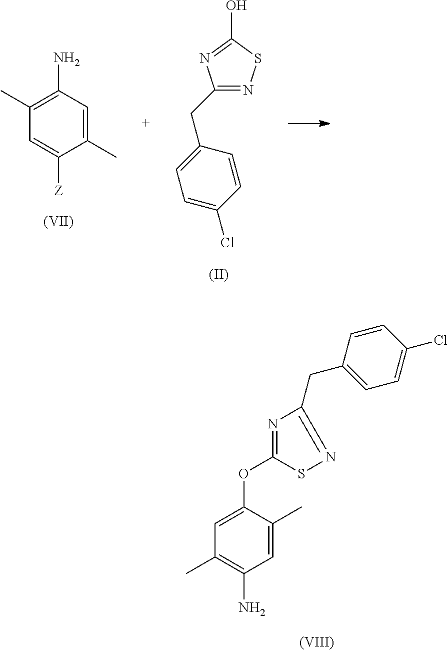
(d) reaction of an aminophenol of the formula (XII) with thiadiazolyl derivatives of the formula (IV) according to the following reaction scheme: 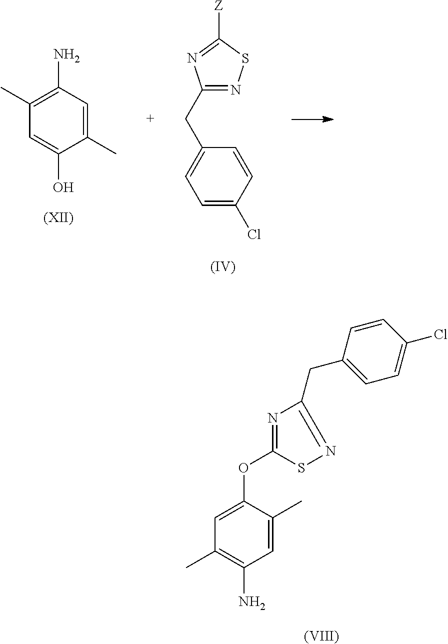
(e) reduction of the nitrophenoxy ether of the formula (VI) to give the aniline ether of the formula (VIII) according to the following reaction scheme: 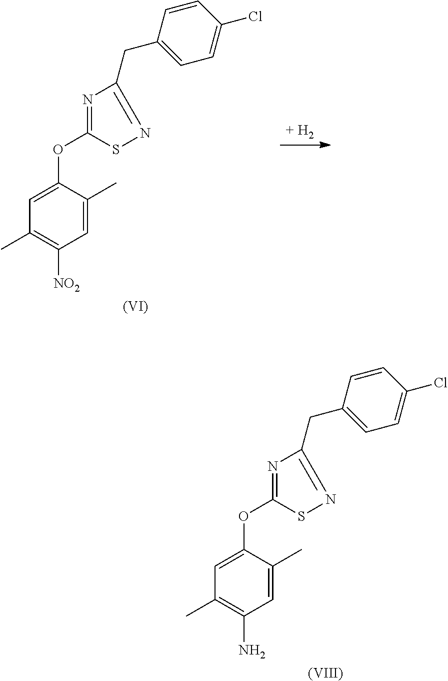
(f) reaction of the aniline ether of the formula (VIII) with
(i) aminoacetals of the formula (XII) or
(ii) an amide of the formula (XIV) or
(iii) an amine of the formula (XV) in the presence of ortho esters of the formula (XVI)
(iv) ortho esters of the formula (XVI) to give imidoformates of the formula (XVIII) and in a second step with methylethylamine (XV)
according to the following reaction scheme: 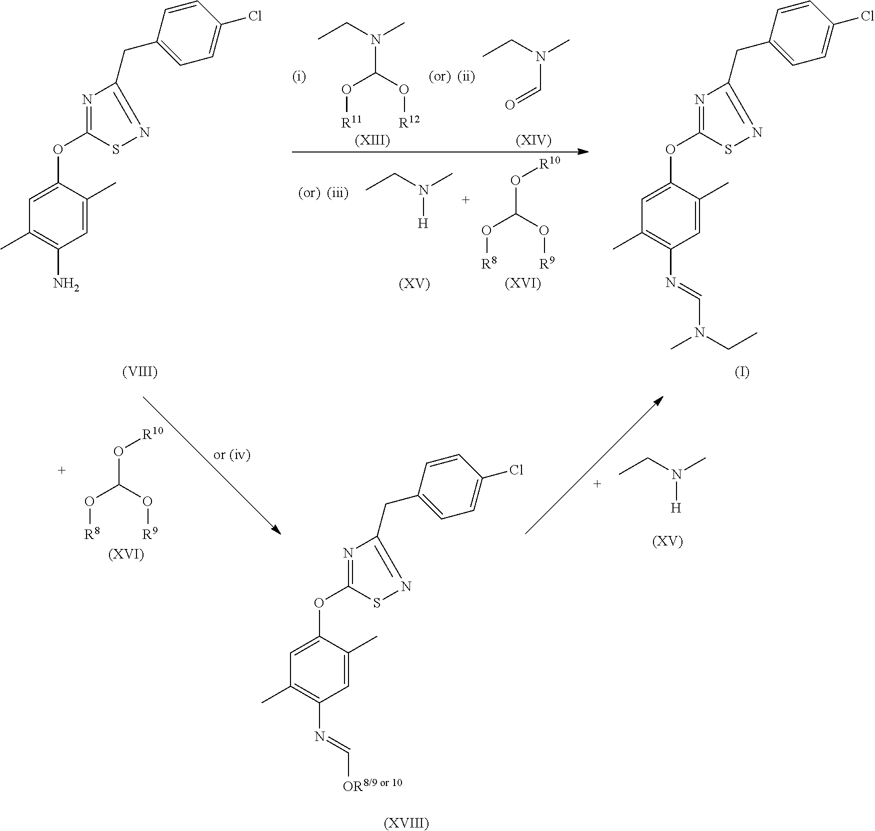
(g) reaction of an aminophenol of the formula (XII) with
(i) aminoacetals of the formula (XIII) or
(ii) an amide of the formula (XIV) or
(iii) an amine of the formula (XV) in the presence of ortho esters of the formula (XVI)
(iv) ortho esters of the formula (XVI) to give imidoformates of the formula (XIX) and in a second step with methylethylamine (XV)
according to the following reaction scheme: 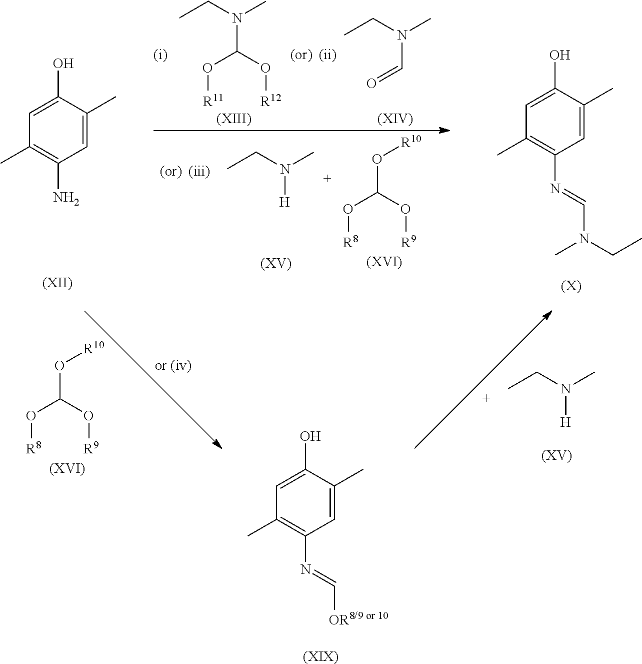
(h) reaction of the aminophenols of the formula (VII) with
(i) aminoacetals of the formula (XIII) or
(ii) an amide of the formula (XIV) or
(iii) an amine of the formula (XV) in the presence of ortho esters of the formula (XVI)
(iv) ortho esters of the formula (XVI) to give imidoformates of the formula (XX) and in a second step with methylethylamine (XV)
according to the following reaction scheme: 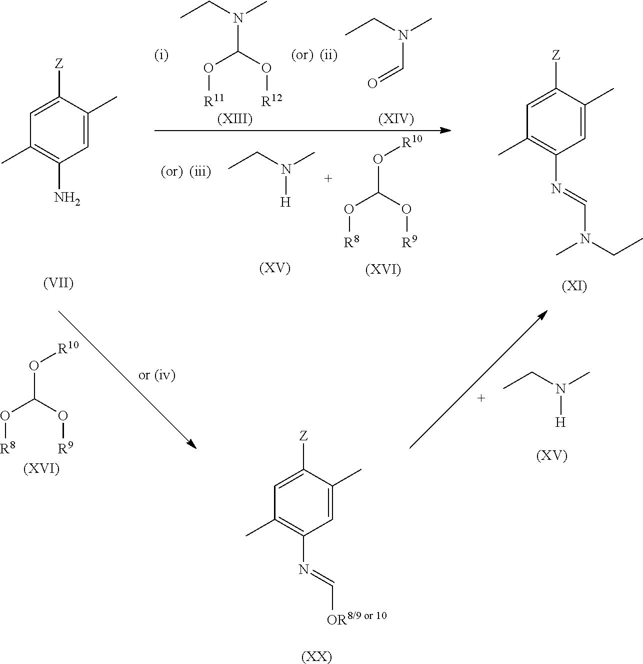
(i) reaction of amidines of the formula (XI) with a thiadiazolyl alcohol of the formula (II) according to the following reaction scheme: 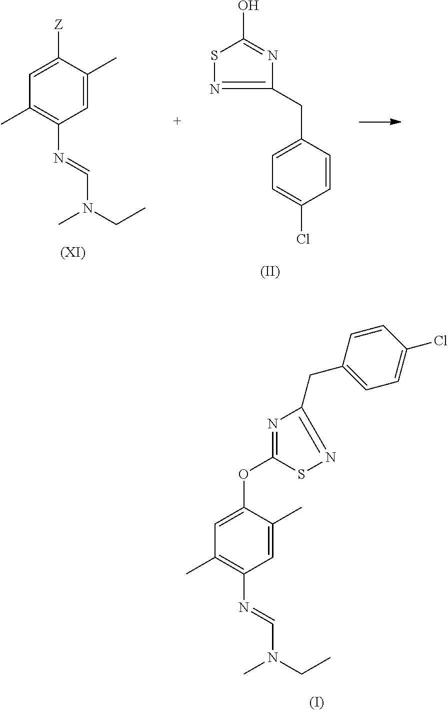
(j) reaction of an amidine of the formula (X) with thiadiazolyl derivatives of the formula (IV) according to the following reaction scheme: 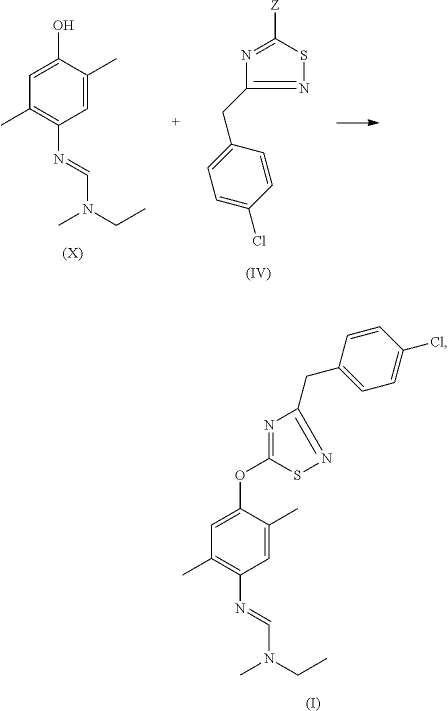
where, in the above schemes,
Z is a leaving group;
and
R⁸ to R¹⁰ independently of one another, are selected from the group consisting of hydrogen, C₁₋₁₂-alkyl, C₂₋₁₂-alkenyl, C₂₋₁₂-alkynyl, C₅₋₁₈-aryl, C₇₋₁₉-arylalkyl or C₇₋₁₉-alkylaryl groups and in each case R⁸ with R⁹, R⁸ with R¹⁰ or R⁹ with R¹⁰, together with the atoms to which they are bonded and optionally with further C, N, O or S atoms, can form a five-, six- or seven-membered ring;
R¹¹ and R¹² independently of one another, are selected from the group consisting of hydrogen, C₁₋₁₂-alkyl, C₂₋₁₂-alkenyl, C₂₋₁₂-alkynyl, C₅₋₁₈-aryl or C₇₋₁₉-arylalkyl groups and, together with the atoms to which they are bonded, can form a five-, six- or seven-membered ring.

2. Thiadiazolyl alcohol of the formula (II)

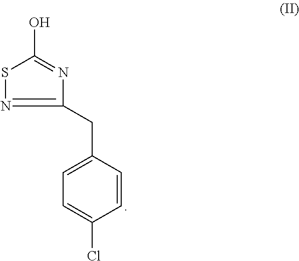

3. Thiadiazolyl derivatives of the formula (IV) 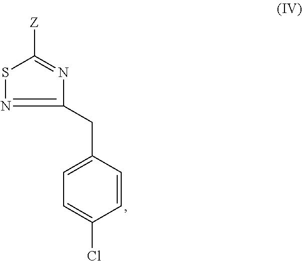
in which Z is a leaving group, selected from the group consisting of halogens, triflate, mesylate, tosylate or SO₂Me.

4. Thiadiazolyl aminophenyl ether of the formula (VIII)

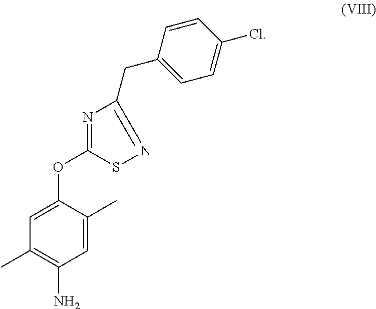

5. Thiadiazolyl nitrophenyl ether of the formula (VI)

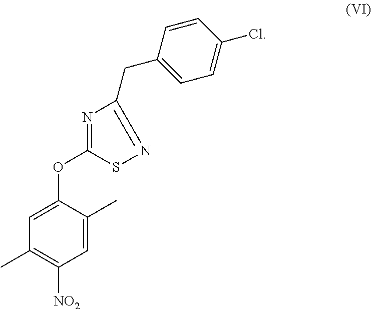

6. Imidoformates of the formula (XVIII) 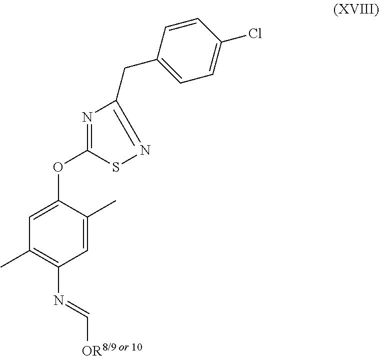
where R⁸, R⁹ and R¹⁰ have the same meaning as given in claim 1.

7. Imidoformates of the formula (XIX) 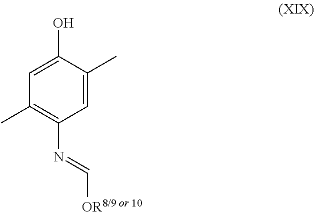
where R⁸, R⁹ and R¹⁰ have the same meaning as given in claim 1.
